(19) United States
(12) Patent Application Publication
(10) Pub. No.: US 2021/0145056 A1
(43) Pub. Date: May 20, 2021

(54) VAPORIZER UNIT FOR A VAPORIZER DEVICE OF AN INHALER

(71) Applicants: VON ERL. GMBH, Hall in Tirol (AT); MCI MANAGEMENT CENTER INNSBRUCK-INTERNATIONALE HOCHSCHULE GMBH, Innsbruck (AT)

(72) Inventors: Reinhard Neuner, Leutasch (AT); Ronald Stärz, Sistrans (AT); Benjamin Hupfauf, Fließ (AT)

(21) Appl. No.: 17/057,635

(22) PCT Filed: Mar. 19, 2019
(86) PCT No.: PCT/AT2019/060091

§ 371 Date: Nov. 20, 2020

(30) Foreign Application Priority Data
May 23, 2018 (AT) A 50418/2018

(51) Int. Cl. A24F 40/44; A24F 40/46; A24F 40/10; A24F 40/70; D01F 6/66; D01F 1/09; D01F 1/10; C08K 3/34; C08K 3/04; C08J 9/26

(52) U.S. Cl. CPC A24F 40/44; C08J 2201/0446; A24F 40/10; A24F 40/70; D01F 6/665; D01F 1/09; D01F 1/10; C08K 3/346; C08K 3/04; C08J 9/26; D10B 2401/022; D10B 2401/16; D10B 2401/10; C08J 2361/16; C08J 2205/05; A24F 40/46

(57) ABSTRACT

The invention relates to a vaporizer unit (21) for a vaporizer device (2) of an inhaler, in particular of an electronic cigarette (1), the vaporizer unit (21) consisting of a material that comprises at least one thermoplastic, preferably a high-performance thermoplastic. The invention also relates to a method for producing such a vaporizer unit (21).

The invention relates to a vaporizer unit with the features of the preamble of claim 1, a vaporizer device with such a vaporizer unit, an inhaler and a mouthpiece for an inhaler with a vaporizer device and a method for producing a vaporizer unit.

Inhalers such as electronic cigarettes, which are often also referred to as electric or electronic cigarettes, usually contain a vaporizer device for vaporizing a liquid substance, also called liquid. Typical vaporizer devices include a vaporizer unit (sometimes also referred to as vaporizer core) to which the liquid can be fed, and a heating device which is arranged in or on the vaporizer unit and which heats the liquid present in the vaporizer unit until it vaporizes. When sucking on a mouthpiece of the inhaler, an air flow is usually created inside the inhaler which is enriched with the vaporized liquid and subsequently the mixture of air and vaporized liquid is inhaled by the user of the inhaler.

Known vaporizer units comprise cotton that is in communication with a liquid that is usually provided in a tank inside the inhaler. A heating device in the form of a heating coil is often wrapped around the vaporizer unit. The vaporizer unit consisting of cotton absorbs the liquid and the heating coil heats the liquid until it vaporizes/evaporates. Cotton is a natural product, the properties of which are subject to large fluctuations in some cases. For example, cotton consists of individual cotton fibers, the fiber lengths, fiber diameters and tensile strengths of which can vary greatly. Accordingly, for a given shape of the vaporizer unit, the absorption capacity of liquid, i.e. how much liquid can be absorbed by the vaporizer unit, varies depending on the cotton used. With regard to the cotton for vaporizer units, it is also the case that there are only very few suppliers on the market and one is therefore completely dependent on the quality of the cotton offered by these suppliers. Moreover, the production of conventional vaporizer units is done predominantly manually, as a result of which vaporizer units with different densities are produced. Furthermore, assembling the vaporizer unit with a heating coil is mostly done manually, so that the heating coils wrap the vaporizer units with different tightness. The result of all this is an unpredictable vaporizer behavior which depends on the specific composition of the cotton and the respective manual manufacture of the vaporizer unit.

The vaporization behavior or the vaporization result of conventional vaporizer units is hardly predictable and, above all, does not remain constant but fluctuates greatly between different vaporizer units manufactured in one series.

It is an object of the invention to provide a vaporizer unit that is improved with respect to the prior art, a vaporizer device with such a vaporizer unit as well as an inhaler and a mouthpiece for an inhaler with such an improved vaporizer device. In particular, a vaporizer unit with a more predictable vaporizer behavior is to be provided. In addition, an improved method for producing a vaporizer unit is to be provided.

This object is achieved by a vaporizer unit with the features of claim 1, a vaporizer device with such a vaporizer unit, an inhaler and a mouthpiece for an inhaler with a vaporizer device and also by methods with the features of claims 21 and 29.

Advantageous embodiments of the invention are defined in the dependent claims.

The invention provides that the vaporizer unit consists of a material that comprises at least one thermoplastic material, preferably a high-performance thermoplastic.

By producing the vaporizer unit from a material comprising at least one thermoplastic material, preferably a high performance thermoplastic, it is possible to produce a plurality of vaporizer units which all have the same material properties. As a result, the vaporizer behavior of the vaporizer units is more predictable and is no longer subject to such large fluctuations.

A high-performance thermoplastic is a thermoplastic material or thermoplastic which has service temperatures in the meaning of the application and allows processing within the meaning of the invention, and has the necessary chemical and mechanical properties which result within the meaning of the application and processing. The service temperature therefore comprises the temperature range which is suitable for the vaporization of the liquid. Furthermore, such a thermoplastic should be able to withstand a time-related load appropriate for the application and processing.

In general, a distinction is made between semi-crystalline and amorphous thermoplastics, wherein a melting point is usually specified for semi-crystalline thermoplastics and a glass transition temperature is specified for amorphous thermoplastics. As soon as the glass transition temperature is exceeded, softening occurs.

Since a component of common liquids for electronic cigarettes is propylene glycol with a vaporization temperature of approx. 188° C., the thermoplastic used (e.g. a polymer) should have a correspondingly higher melting temperature or a glass transition temperature of approx. >88° C. Since the vaporizer unit of an electronic cigarette does not have to withstand any mechanical load, softening may be allowed, which results in a requirement with regard to the glass transition temperature for the thermoplastic that is lower than the requirement with respect to the vaporization temperature. Another application uses a liquid comprising glycerine which vaporizes/evaporates at approx. 290° C. The corresponding thermoplastic would therefore preferably have a melting temperature of >290° C. or a glass transition temperature of about >190° C. In a particularly preferred case, a liquid mixture is used which vaporizes at approx. 250° C. A corresponding thermoplastic, for example in the form of a partially crystalline polymer, would preferably have a melting temperature >250° C. and a corresponding thermoplastic, for example in the form of an amorphous polymer, would preferably have a glass transition temperature of approx. >150° C.

It can therefore preferably be provided that a melting temperature of the at least one thermoplastic material is higher than about 180° C., preferably higher than about 250° C.

Likewise, it can preferably be provided that a glass transition temperature of the at least one thermoplastic material is higher than about 80° C., preferably higher than about 150° C.

For example, a thermoplastic which meets the above-mentioned requirements can be a polyaryl-ether-ketone or polyaryletherketone (PAEK), preferably poly-ether-ether-ketone or polyetheretherketone (PEEK).

Other suitable thermoplastics are poly-sulfones such as polysulfone (PSU), polyethersulfone (PES), polyphenylsulfone (PPSU) or polyimides (PI) such as polyetherimide (PEI), polyamide-imide (PAI) or polyphenylene sulfide (PPS), polyamide (PA), polyphenylene ether (PPE), polyphthalamide (PPA). In addition, co-polymers or mixtures thereof, as well as filled, unfilled or fiber-reinforced thermoplastics can be used.

Therefore, it can preferably be provided that the material of the vaporizer unit comprises at least one of the following thermoplastic materials: a polyaryletherketone (PAEK), preferably polyetheretherketone (PEEK); a poly sulfone, preferably polysulfone (PSU) and/or polyethersulfone (PES) and/or polyphenylsulfone (PPSU); a polyimide (PI), preferably polyetherimide (PEI) and/or polyamide-imide (PAI); polyphenylene sulfide (PPS); polyamide (PA); polyphenylene ether (PPE); polyphthalamide (PPA).

Particularly preferably, it can be provided that the vaporizer unit consists substantially entirely of at least one polyaryletherketone, preferably polyetheretherketone. However, it is also possible that the material of the vaporizer unit contains additives and/or electrically conductive additives in addition to a polyaryletherketone.

Polyaryletherketones, abbreviated PAEK, are high-temperature-resistant thermoplastics, also called high-performance thermoplastics. They have outstanding mechanical stability as well as chemical resistance to a multiplicity of solvents. A widespread representative of these high-temperature-resistant materials is polyetheretherketone, abbreviated PEEK. PEEK only melts at a relatively high temperature of 335° C. and can be molded using the injection molding method or per extruder. In the solid state, PEEK can be easily processed further (e.g. cut or milled). The use of polyaryletherketones as material for the vaporizer unit makes it possible to produce a plurality of vaporizer units, all of which have the same material properties. As a result, the behavior of the vaporizer unit is more predictable and no longer subject of large fluctuations.

According to a particularly preferred embodiment, it can be provided that the vaporizer unit is porous. A porous structure of the vaporizer unit, thus structure with cavities, increases the absorption capacity of the vaporizer unit for the liquid to be supplied to the vaporizer unit during use.

Preferably, it can be provided that the vaporizer unit has an open porosity of about 30% to 96%, preferably of about 50% to 95%, particularly preferably of about 65% to 94%. The open porosity takes into account the cavities in the structure of the vaporizer unit which are connected to each other and to the environment and thus represents the percentage of these cavities of the total volume of the vaporizer unit.

Particularly preferably, it can be provided that the vaporizer unit is self-supporting. A statically self-supporting design of the vaporizer unit facilitates the processing (e.g. cutting or milling) of the vaporizer unit and the installation thereof in a vaporizer device.

Preferably, it can be provided that the material of the vaporizer unit comprises at least one additive.

Thus, an additive such as diatomaceous earth or alumina can be added to the material of the vaporizer unit. Such solids, which can preferably be porous in themselves, remain in the structure of the vaporizer unit during the production of the vaporizer unit and can partially prevent the bonding of the thermoplastics of the vaporizer unit, which facilitates the porosity of the vaporizer unit.

Further examples of additives are: fluids, preferably water and/or isopropanol; blowing agents, preferably NaHCO₃; soluble solids, preferably soluble salts (e.g. chlorides, carbonates or halides) and/or NaCl, Na₂CO₃, CaCl₂), KCl, MgCl₂.

It can also be provided that the material of the vaporizer unit comprises at least one electrically conductive additive, the at least one electrically conductive additive preferably being a metal, graphite, graphene, silicon carbide or activated carbon. By adding an electrically conductive additive to the material of the vaporizer unit, the vaporizer unit itself is electrically conductive, whereby it can itself constitute a heating resistor and thus itself also a heating device.

If the at least one electrically conductive additive has a good thermal conductivity, such as metals or silicon carbide, the heat conduction into the liquid to be vaporized can be facilitated.

According to a preferred embodiment, it can be provided that the vaporizer unit is formed to be cylindrical, preferably circular cylindrical.

It can preferably be provided that a surface of the vaporizer is provided with at least one electrically conductive coating, at least in certain sections. By providing at least one electrically conductive coating on a surface of the vaporizer unit, a separate heating device in the form of a heating coil which is wound around a vaporizer unit and makes contact with the vaporizer unit, which contact cannot be precisely determined, can be dispensed with. Instead, the vaporizer unit has at least one coating which is in optimum contact with the vaporizer unit and can serve as a heating device. This way, the behavior of the vaporizer unit is more predictable and is no longer subject to large fluctuations.

According to a preferred embodiment, it can also be provided that the vaporizer unit is configured, at least in certain sections, preferably substantially completely, as a textile structure, preferably as a flat or spatial fabric, the textile structure comprising at least one fiber and/or at least one filament comprising the at least one thermoplastic material.

The textile structure can also be a warp-knitted fabric, knitted fabric, meshwork, stitch-bonded fabric, non-woven, felt or, for example, a textile tube.

In general, the textile structure can be designed to be flat (for example, like a filter) or cylindrical or round (for example, like a cord).

Preferably, it can be provided that the at least one fiber and/or the at least one filament consist or consists substantially entirely of at least one polyaryletherketone, preferably polyetheretherketone.

It can be provided that the at least one fiber and/or the at least one filament have or has a diameter of less than 1000 μm, preferably less than 500 μm, particularly preferably less than 300 μm. In a preferred embodiment, the diameter of the at least one fiber and/or the at least one filament can be smaller than 50 μm, for example 38 μm.

It can also be provided that the textile structure is composed of at least one thread and/or yarn comprising the at least one fiber and/or the at least one filament.

It can preferably be provided that the textile structure is formed to be wick-like. In this case, the textile structure is particularly suitable for absorbing and transporting a liquid for an electronic cigarette. The open porosity of the textile structure can be changed in particular by the number, diameter and type of processing the fibers and/or filaments, in order to meet the requirements as a wick (porous structure). Possible types of processing are, for example: spinning, weaving, warp-knitting, knitting, braiding, felting, joining.

It can be provided that the textile structure contains at least one additive. Possible additives for the material of the vaporizer unit have already been mentioned. The at least one additive can already be contained in the at least one fiber or the at least one filament (e.g. filled PEEK). However, it can also be processed, for example woven, in the form of an additive fiber or an additive filament with the at least one fiber and/or the at least one filament. Examples of such additive fibers or additive filaments are metal wires or metal bands as well as graphite, glass or basalt filaments. Additive fibers or additive filaments can also serve as heating devices. Preferably, the textile structure can be a PEEK fabric containing at least one additive.

The textile structure can be formed, for example, as a flat fabric. The flat fabric can be rolled, folded or stacked. To obtain a round geometry, common weaving methods can be used. Cutting the textile structure to size can be carried out with a tempered blade thereby fusing and fixing the ends of the fibers and/or filaments.

The textile structure can also form a kind of sheath around a core material. The core material in turn can comprise at least one thermoplastic material, preferably a high-performance thermoplastic. Examples of suitable thermoplastics and high-performance thermoplastics have already been given. For example, a textile structure in the form of a PEEK fabric could be wrapped or braided around a core material. In this way, the core material would be shielded from temperature peaks, such as the ones that can occur in a vaporizer of an electronic cigarette.

Protection is also sought for a vaporizer device for an inhaler according to claim 16, an inhaler according to claim 19 and a mouthpiece for an inhaler according to claim 20. Advantageous embodiments are specified in the respective dependent claims.

It can preferably be provided that a vaporizer device is provided with a heating device, the heating device being arranged in or on the vaporizer unit. The heating device can be an electrically conductive coating which is arranged on a surface of the vaporizer unit. It can also be an electrically conductive wire, for example, which is integrated into the vaporizer body or wrapped around the vaporizer unit.

According to a preferred design variant, it can be provided that the vaporizer device comprises a receiving chamber for a liquid, with the vaporizer unit being in communication with the liquid.

Moreover, protection is sought for a method for producing a vaporizer unit with the features of claim 21. Advantageous embodiments are specified in the claims dependent thereon.

In the proposed method, a mixture comprising at least one thermoplastic material, preferably a high-performance thermoplastic, is provided, the mixture being heated until the mixture is at least superficially melted or a melt is formed, at least one vaporizer unit being formed from the at least superficially melted mixture or the melt.

The mixture constitutes the material of the vaporizer unit.

Preferably, the mixture can comprise at least one of the following thermoplastic materials: a polyaryletherketone (PAEK), preferably polyetheretherketone (PEEK); a poly sulfone, preferably polysulfone (PSU) and/or polyethersulfone (PES) and/or polyphenylsulfone (PPSU); a polyimide (PI), preferably polyether-imide (PEI) and/or polyimide-imide (PAI); polyphenylenesulfide (PPS); polyimide (PA); polyphenyleneether (PPE); polyphthalamide (PPA).

It can be provided that the mixture comprises at least one electrically conductive additive—preferably a metal, graphite, graphene, silicon carbide or activated carbon—which is mixed with the at least one thermoplastic material to provide the mixture.

The electrically conductive additives can preferably have good thermal conductivity, such as metals or silicon carbide (SiC).

Preferably, the particle diameters of the admixed electrically conductive additives are smaller than about 2000 μm, preferably smaller than about 1000 μm, particularly preferably smaller than about 500 μm.

Adding preferably powdery, electrically conductive additives to the mixture results in the finished vaporizer unit being electrically conductive in itself, which means that the vaporizer itself constitutes a kind of heating resistor that heats the entire vaporizer unit when an electrical voltage is applied.

It can be provided that the mixture comprises at least one additive which is mixed with the at least one thermoplastic to provide the mixture.

These additives can be solids, which can also be inherently porous. These additives can partially prevent the thermoplastic material from bonding during the production process of the vaporizer unit, which can affect the porosity of the vaporizer unit. The additives can remain in the structure of the finished vaporizer unit.

Examples of additives are: diatomaceous earth; alumina; fluids, preferably water and/or isopropanol; blowing agents, preferably NaHCO₃; soluble solids, preferably soluble salts (e.g. chlorides, carbonates or halides) and/or NaCl, Na₂CO₃, CaCl₂), KCl, MgCl₂.

Diatomaceous earth as an additive has the advantage that it is inherently porous and thus contributes to an increased porosity of the vaporizer unit. With low mass fractions of such an additive, of e.g. 20%, sufficient porosity can already be produced.

Preferably, the particle diameters of the added additives are smaller than about 2000 μm, preferably smaller than about 1000 μm, and particularly preferably smaller than about 500 μm.

It can also be provided that at least one fluid, such as water or isopropanol, is added to the mixture. Such a fluid facilitates the mixing of the mixture and further processing since with the addition of a fluid, the bulk volume can be reduced and a pasty consistency of the mixture can be obtained. If the added fluid is not removed (e.g. by drying) before the mixture is superficially partially melted or melted, it can act as a blowing agent during the heating of the mixture and cause additional porosity. For example, NaHCO₃ (in the trade often referred to as “sodium bicarbonate”) can also be added as a blowing agent.

It can preferably be provided that the mixture comprises at least one soluble solid, preferably a salt, the at least one soluble solid being mixed with the at least one thermoplastic material to provide the mixture, wherein, after molding the at least one vaporizer unit, the at least one soluble solid is at least partially, preferably substantially entirely, dissolved from the vaporizer unit.

As a result, the structure of the vaporizer unit contains soluble solid particles (e.g. in the form of salt particles) which influence the porosity of the vaporizer unit. If the at least one soluble solid is a soluble salt, it can be dissolved out of the vaporizer unit after molding using a suitable solvent such as water (H₂O). This creates cavities in the structure of the vaporizer unit which are connected to each other and to the environment and which influence the open porosity of the vaporizer unit. The soluble salts can be chlorides, carbonates or halides. Preferably, NaCl, Na₂CO₃, CaCl₂), KCl, MgCl₂ in the form of soluble solid particles can be added to the mixture.

Preferably, the particle diameters of the added soluble solid particles are smaller than about 2000 μm, preferably smaller than about 1000 μm, particularly preferably smaller than about 500 μm.

According to a preferred embodiment it can be provided that the at least one thermoplastic material is a powdery or flaky polyaryletherketone, preferably polyetheretherketone. Preferably, it can be provided that the mass fraction of the powdery or flaky polyaryletherketone in the mixture is about 4% to 65%, preferably about 5% to 55%, particularly preferably about 6% to 45%.

A powdery polyetheretherketone with particle diameters smaller than about 5000 μm can preferably be used for the mixture. Preferably, the particle diameter is less than about 1000 μm. Commercially available polyetheretherketone powders often include PEEK particles with particle diameters that are distributed over a wide range. For such powders, the specification in terms of an average particle diameter or the average grain size is often provided. Preferably, a PEEK powder with an average grain size of less than about 100 μm is used. A suitable PEEK powder is commercially available under the name VESTAKEEP® 2000 FP, for example. This powder has an average grain size of about 50 μm and a melt volume-flow rate (MVR) of 70 cm³/10 min (test temperature 380° C., mass 5 kg).

It may also be provided that the mixture comprises a flaky polyaryletherketone. Here, the shape of the flakes may well deviate from the spherical shape. Preferably, the flakes have a diameter of less than about 5000 μm and a thickness of less than about 200 μm.

According to a particularly preferred embodiment variant, it can be provided that powdered polyetheretherketone is mixed with sodium chloride, wherein preferably the mass fraction of the powdered polyetheretherketone in the mixture is about 4% to 65%, particularly preferably about 5% to 55%, more particularly preferably about 6% to 45%, an average grain size of the powdered polyetheretherketone preferably being smaller than about 1000 μm, particularly preferably smaller than about 100 μm.

Preferably, it can be provided that the mixture is heated to a maximum temperature between about 335° C. and about 450° C., preferably to about 370° C., the mixture preferably being exposed to the maximum temperature for a residence time of up to 4 h, particularly preferably about 0.5 h to 1.5 h.

Since the melting temperature of PEEK is 335° C. and the decomposition temperature is about 450° C., the mixture should be heated to a maximum temperature above the melting temperature of PEEK and below the decomposition temperature of PEEK.

Experiments carried out by the applicant have shown that a maximum temperature of about 370° C. results in a melt that can be easily processed further.

Preferably, the heating of the mixture is so high and/or long enough that the mixture starts to melt at least on the surface or forms a melt. Depending on the further processing method, the residence time during heating can range from only about 1 s (e.g. in a continuous process) to about 4 h.

The vaporizer units can be molded using the following known forming processes: Batch (flat production, the molds are later drilled, punched or cut; with or without integral heating element), extrusion (as string or tube; with or without integral heating element), sintering or injection molding.

If a soluble solid (such as a salt) was added to the mixture prior to heating and molding, it can be provided that after molding the vaporizer unit and, if necessary, after cooling the vaporizer unit, the soluble solid is dissolved out of the vaporizer unit with a suitable solvent (e.g. water) as far as possible to obtain the desired porosity of the vaporizer unit. Thus, the vaporizer unit can have an open porosity of about 30% to 96%, preferably of about 50% to 95%, particularly preferably of about 65% to 94%.

Examples of vaporizer units produced according to the proposed method are given below.

### EXAMPLE 1

The mixture contains PEEK with a mass fraction of 8% and NaCl with a mass fraction of 92%. The particle diameter of the PEEK powder ranges from about 4 μm to about 832 μm, with an average particle diameter of about 70 μm. The particle diameter of the NaCl powder ranges from about 189 μm to about 1200 μm, with an average particle diameter of about 479 μm. The mixture is heated in a furnace. The furnace temperature is about 370° C. and the furnace residence time is about 1 h. After cooling and solidification of the melt, the NaCl is dissolved out with water. This results in an open porosity of about 91%.

### EXAMPLE 2

The mixture contains PEEK with a mass fraction of 20% and NaCl with a mass fraction of 80%. The particle diameter of the PEEK powder ranges from about 4 μm to about 832 μm, with an average particle diameter of about 70 μm. The particle diameter of the NaCl powder ranges from about 132 μm to about 692 μm, with an average particle diameter of about 293 μm. The mixture is heated in a furnace. The furnace temperature is about 370° C. and the furnace residence time is about 1 h. After cooling and solidification of the melt, the NaCl is dissolved out with water. This results in an open porosity of about 73%.

### EXAMPLE 3

The mixture contains PEEK with a mass fraction of 15% and NaCl with a mass fraction of 85%. The particle diameter of the PEEK powder ranges from about 4 μm to about 832 μm, with an average particle diameter of about 70 μm. The particle diameter of the NaCl powder ranges from about 0.9 μm to about 437 μm, with an average particle diameter of about 293 μm. The mixture is heated in a furnace. The furnace temperature is about 370° C. and the furnace residence time is about 1 h. After cooling and solidification of the melt, the NaCl is dissolved out with water. This results in an open porosity of about 85%.

### EXAMPLE 4

The mixture contains PEEK with a mass fraction of 23%, diatomaceous earth with a mass fraction of 23% and H₂O with a mass fraction of 54%. The particle diameter of the PEEK powder ranges from about 4 μm to about 832 μm, with an average particle diameter of about 70 μm. The particle diameter of the diatomaceous earth powder ranges from about 0.8 μm to about 331 μm, with an average particle diameter of about 9 μm. The mixture is first heated to 105° C. for a period of 12 h, which dries the mixture. The heating of the mixture is then continued in a furnace. The furnace temperature is about 370° C. and the furnace residence time is about 1 h. This results in an open porosity of about 85%.

### EXAMPLE 5

The mixture contains PEEK with a mass fraction of 40% and graphite with a mass fraction of 60%. The particle diameter of the PEEK powder ranges from about 4 μm to about 832 μm, with an average particle diameter of about 70 μm. The particle diameter of the graphite powder ranges from about 1 μm to about 631 μm, with an average particle diameter of about 18 μm. The mixture is heated in a furnace. The furnace temperature is about 370° C. and the furnace residence time is about 1 h. This results in an open porosity of about 57%.

Protection is also sought for a method for producing a vaporizer unit with the features of claim 29. Advantageous embodiments are specified in the claims dependent thereon.

In the proposed method, at least one fiber and/or at least one filament comprising the at least one thermoplastic material are/is processed to form a textile structure, preferably a flat or spatial fabric, the vaporizer unit or a part thereof being formed from the textile structure.

It can be provided that a thread and/or yarn comprising the at least one fiber and/or the at least one filament are/is processed to form the textile structure.

Preferably, it can be provided that processing is carried out using at least one of the following methods: spinning, weaving, warp-knitting, knitting, braiding, felting, joining.

It can be provided that during the step of molding the vaporizer unit, the textile structure is cut to size with a tempered blade.

In general, it can be provided according to a preferred embodiment that at least one electrically conductive coating is applied onto a surface of the vaporizer unit, at least in certain sections. The at least one electrically conductive coating can serve as a heating device.

Preferably, it can be provided that the at least one electrically conductive coating is applied galvanically onto the surface of the vaporizer unit. This can be done by means of electroplating techniques known per se. Any metal powder particles present in the vaporizer unit can serve as “nuclei” for electroplating.

It may also be provided that the at least one electrically conductive coating is vapor-deposited onto the surface of the vaporizer unit.

Further details and advantages of the present invention are explained based on the following description of the figures. In the figures:

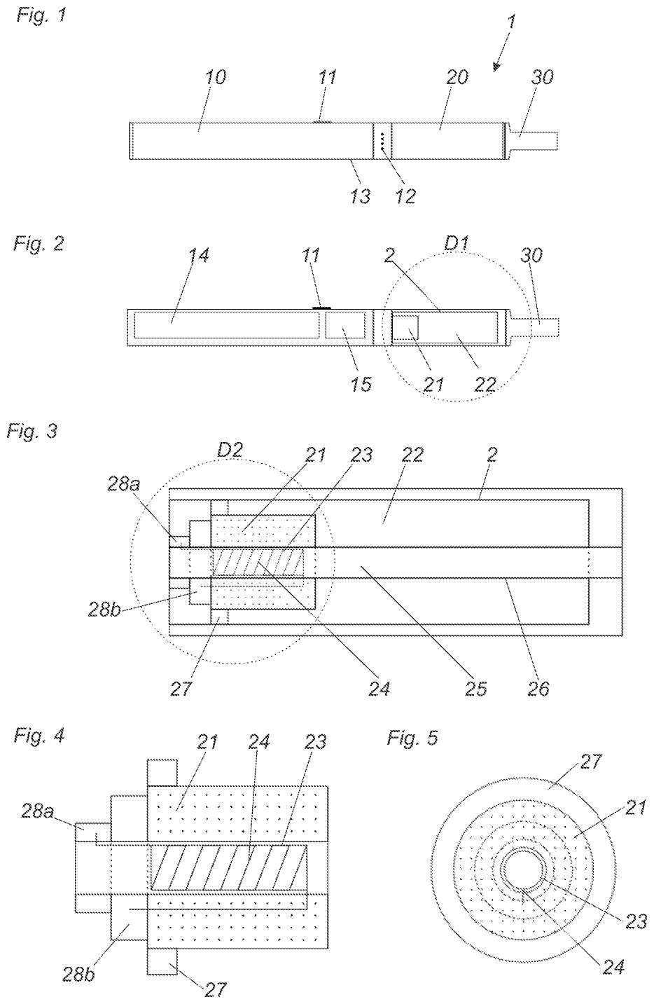

FIG. 1 shows an exemplary embodiment of an inhaler in the form of an electronic cigarette in a front view,

FIG. 2 shows a sectional view through an electronic cigarette according to FIG. 1,

FIG. 3 shows an enlarged detailed view of an area of FIG. 2,

FIG. 4 shows an enlarged detailed view of an area of FIG. 3,

FIG. 5 shows a side view of the vaporizer unit of FIG. 4,

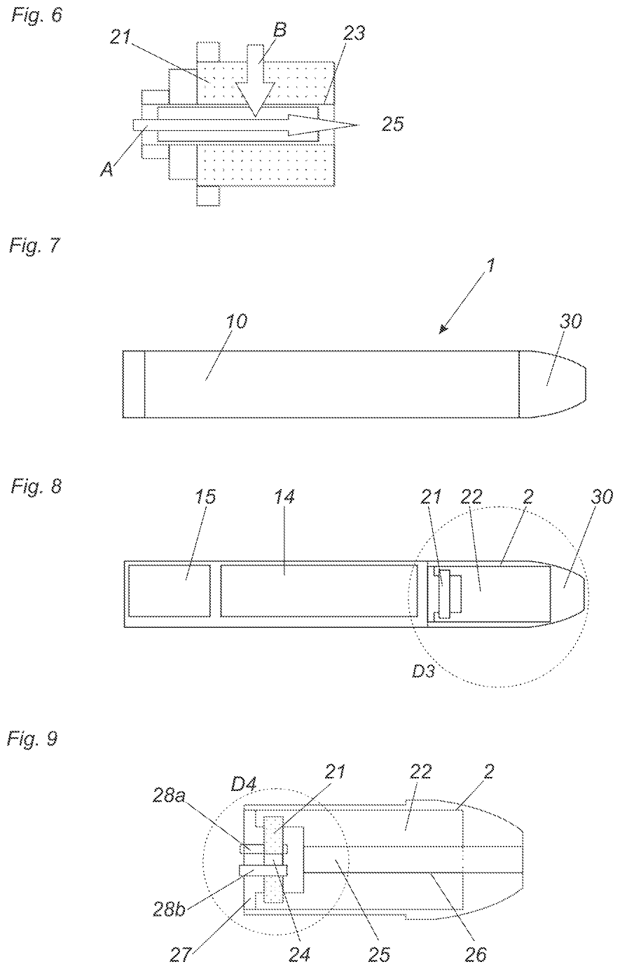

FIG. 6 shows a schematic illustration according to FIG. 4,

FIG. 7 shows another exemplary embodiment of an inhaler in the form of an electronic cigarette in a front view,

FIG. 8 shows a sectional view through an electronic cigarette according to FIG. 7,

FIG. 9 shows an enlarged detailed view of an area of FIG. 8,

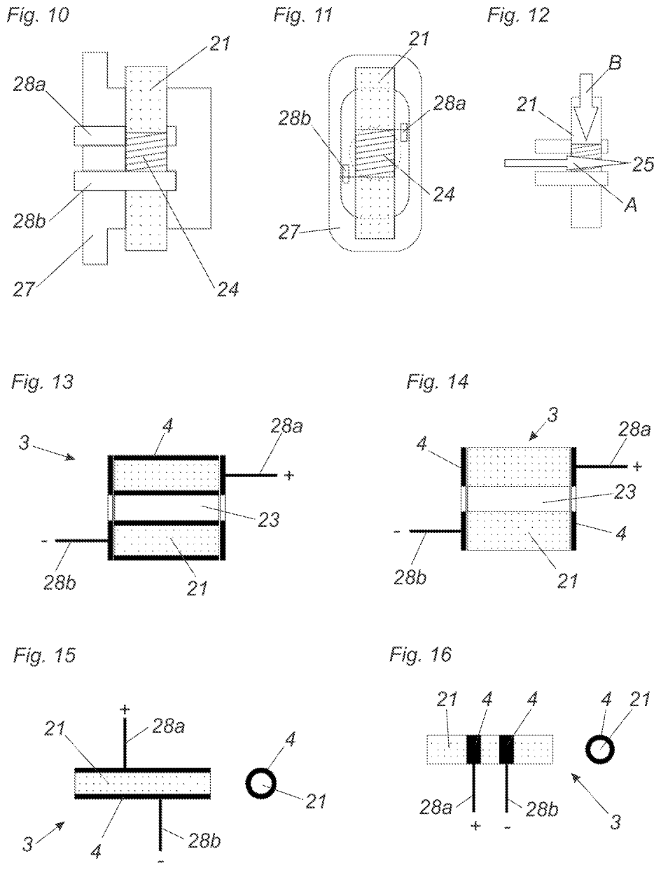

FIG. 10 shows an enlarged detailed view of an area of FIG. 9,

FIG. 11 shows a side view of the vaporizer unit of FIG. 10,

FIG. 12 shows a schematic illustration according to FIG. 9,

FIGS. 13-17 show exemplary embodiments of proposed vaporizer units,

FIG. 18 shows an exemplary embodiment of a vaporizer device, and

FIG. 19 shows an exemplary embodiment of a vaporizer unit formed as a textile structure.

FIG. 1 shows an exemplary embodiment of an inhaler in the form of an electronic cigarette 1 in a front view.

The electronic cigarette 1 comprises a first housing part 10, in which an energy storage in the form of a secondary battery 14, which is not visible in this illustration, and a control electronics 15 of the electronic cigarette 1, which is also not visible here, are accommodated. Furthermore, a control element 11 in the form of a push button for operating the electronic cigarette 1 and a charging connection 13 for charging the secondary battery 14 are attached to the first housing part 10. The first housing part 10 also has an air inlet 12 (here in the form of a plurality of holes arranged one above the other in the housing) through which ambient air can be introduced into the interior of the electronic cigarette 1 by sucking at a mouthpiece 30 of the electronic cigarette 1.

The electronic cigarette 1 furthermore comprises a second housing part 20 which can be screwed to the first housing part 10. The second housing part 20 accommodates a vaporizer device 2 for vaporizing a liquid, which is not visible in this illustration. A mouthpiece 30, through which vaporized liquid can be removed when the electronic cigarette 1 is activated, is arranged on the second housing part 20.

FIG. 2 shows a schematic sectional view through an electronic cigarette 1 according to FIG. 1. Particularly visible in this illustration are the secondary battery 14 and the control electronics 15 which are located inside the first housing part 10. Also visible is the vaporizer device 2, which is arranged in the second housing part 20. The vaporizer device 2 comprises a vaporizer unit 21 and a receiving chamber 22 in the form of a tank containing liquid to be vaporized. The vaporizer unit 21 projects into the receiving chamber 22 and is in communication with the liquid inside.

FIG. 3 shows an enlarged detailed view of the area marked and designated by D1 in FIG. 2.

The vaporizer unit 21 protrudes into the receiving chamber 22 containing the liquid and, together with a circumferential seal 27 arranged around an end region of the vaporizer unit 21, delimits the receiving chamber 22. The vaporizer unit 21 is self-supporting and, in this example, is made substantially entirely of PEEK. The vaporizer unit 21 has an open porosity of about 80%.

A tubular channel 26 which also delimits the receiving chamber 22 runs through the vaporizer unit 21 and the receiving chamber 22 so that the liquid cannot flow into the interior of channel 26. In this example, the vaporizer unit 21 is circular-cylindrical and a portion of the channel 26 in the vaporizer unit 21 is formed as a through-hole 23 passing through the vaporizer unit 21 in a longitudinal direction. Inside the vaporizer unit 21, in this example inside the through-hole 23 passing through the vaporizer unit 21, a heating device 24 in the form of a heating coil is arranged. The heating device 24 is connected to the control electronics 15 via electrical contacts 28a (e.g. a positive voltage connection) and 28b (e.g. a negative voltage connection). When actuating the electric cigarette 1 via the control element 11, current is conducted via the control electronics 15 from the secondary battery 14 into the heating device 24 via the electrical contacts 28a, 28b, thereby heating the heating device 24. Due to the porosity of the vaporizer unit 21, liquid, with which the vaporizer unit 21 is in communication, enters the vaporizer unit 21. The heating of the heating device 24 results in vaporization of the liquid in the vaporizer unit 21. By the user sucking on the mouthpiece 30 of the electronic cigarette 1, air is sucked from outside the electronic cigarette 1 through the air inlet 12 into the interior of the electronic cigarette 1. The air flows through the channel 26 with the air absorbing the vaporized liquid. The mixture 25 of air and vaporized liquid continues to flow through the channel 26 and leaves the electronic cigarette 1 through the mouthpiece 30.

FIG. 4 shows an enlarged detailed view of the area marked and designated by D2 in FIG. 3. Here, the through-hole 23 in the vaporizer unit 21, which forms a portion of the channel 26, is clearly visible. The heating device 24 is arranged in the through-hole 23 and electrically contacted by two electrical contacts 28a, 28b. A circumferential seal 27 is arranged at an end area of the vaporizer unit 21 so that when the vaporizer unit 21 with the circumferential seal 27 is inserted into the receiving chamber 22, the receiving chamber 22 is delimited and sealed at the end face.

FIG. 5 shows a side view of the vaporizer unit 21 in FIG. 4.

FIG. 6 shows a schematic illustration according to FIG. 4 in which in particular the flow of air A, vaporized liquid B and mixture 25 of air A and vaporized liquid B through the through-hole 23 of the vaporizer unit 21 is illustrated.

FIG. 7 shows another exemplary embodiment of an inhaler in the form of an electronic cigarette 1 in a front view. The electronic cigarette 1 consists of a first housing part 10 and a mouthpiece 30 which can be inserted into the first housing part 10. An air inlet 12, which is not visible here, through which air from the environment can enter the interior of the electronic cigarette 1 when the electronic cigarette 1 is operated, is arranged on the first housing part 10.

FIG. 8 shows a schematic sectional view through an electronic cigarette 1 according to FIG. 7. Visible in this illustration are in particular the secondary battery 14 and the control electronics 15, which are arranged inside the first housing part 10. Also visible is the vaporizer device 2 which is arranged in the removable mouthpiece 30. The vaporizer device 2 comprises a vaporizer unit 21 and a receiving chamber 22 in the form of a tank containing liquid to be vaporized. The vaporizer unit 21 is formed to be substantially circular-cylindrical and protrudes with both end portions into the receiving chamber 22 so that it is in communication with the liquid in the receiving chamber 22 via both end regions.

FIG. 9 shows an enlarged detailed view of the area marked and designated by D3 in FIG. 8.

The vaporizer unit 21 is self-supporting and in this example consists substantially entirely of PEEK. The vaporizer unit 21 has an open porosity of about 75%. Both end regions of the vaporizer unit 21 protrude into the receiving chamber 22 which contains the liquid. Due to the porosity of the vaporizer unit 21, liquid can thus penetrate from the receiving chamber 22 into the cavities of the vaporizer unit 21. The vaporizer unit 21 is arranged at a seal 27 in the form of a sealing lid which delimits the receiving chamber 22.

A tubular channel 26 which also delimits the cavity 22 runs through the cavity 22 thereby preventing the liquid from flowing into the interior of the channel 26. The vaporizer unit 21 is arranged in front of an orifice of the channel 26. A heating device 24 in the form of a heating coil which, in this example, is wrapped around a portion of the vaporizer unit 21 is arranged on the vaporizer unit 21. The heating device 24 is connected to the control electronics 15 via electrical contacts 28a (e.g. a positive voltage connection) and 28b (e.g. a negative voltage connection).

The electric cigarette 1 of this example can be activated by a vacuum sensor, which is not illustrated, which is connected to the control electronics 15. By the user sucking on the mouthpiece 30 of the electronic cigarette 1, air from outside the electronic cigarette 1 is sucked through the air inlet 12 into the interior of the electronic cigarette 1. Furthermore, when sucking on the mouthpiece 30, a negative pressure is created inside the electronic cigarette 1 which is detected by the negative pressure sensor, whereupon the electronic cigarette 1 is activated by the control electronics 15. In doing so, current is conducted via the control electronics 15 from the secondary battery 14 to the heating device 24 via the electrical contacts 28a, 28b thereby heating up the heating device 24. Due to the porosity of the vaporizer unit 21, liquid, with which the vaporizer unit 21 is in communication, enters the vaporizer unit 21. Heating up the heating device 24 results in that the liquid vaporizes in the vaporizer unit 21. The sucked-in air flows through or around the vaporizer unit 21 with the air absorbing the vaporized liquid. The mixture 25 of air and vaporized liquid flows into the orifice of the channel 26 and further through the channel 26 and leaves the electronic cigarette 1 through the mouthpiece 30.

FIG. 10 shows an enlarged detailed view of the area marked and designated by D4 in FIG. 9. Here, the vaporizer unit 21, around which the heating device 24 is wrapped in the form of a heating coil, is clearly visible. The heating device 24 is electrically contacted by two electrical contacts 28a, 28b which extend up to the seal 27 in the form of a seal cover and protrude slightly beyond the seal cover to simplify contacting. The shape of the seal cover corresponds to the shape of an end face of the receiving chamber 22 so that when the seal cover is placed on the receiving chamber 22, the receiving chamber 22 is delimited and sealed at the end face.

FIG. 11 shows a side view of the vaporizer unit 21 in FIG. 10.

FIG. 12 shows a schematic illustration of a vaporizer unit 21 according to FIG. 9 in which in particular the flow of air A, vaporized liquid B and mixture 25 of air A and vaporized liquid B is illustrated.

FIG. 13 shows a proposed vaporizer unit 21 in a longitudinal section. The vaporizer unit 21 is formed to circular cylindrical and has a through-hole 23 running therethrough so that the vaporizer unit 21 takes on the overall shape of a round tube. In this example, the entire surface 3 of the vaporizer unit 21—thus, the inner wall of the through-hole 23, the outer surface of the vaporizer unit 21 and the end faces of the vaporizer unit 21—is provided with an electrically conductive coating 4. In this example, the electrically conductive coating 4 constitutes a heating device 24 which can be connected to a corresponding power supply via electrical contacts 28a (e.g. a positive voltage connection) and 28b (e.g. a negative voltage connection).

FIG. 14 shows another exemplary embodiment of a proposed vaporizer unit 21 in a longitudinal section. This vaporizer unit 21 is also formed to be circular tube-shaped, but not the entire surface 3 of the vaporizer unit 21 is coated with an electrically conductive coating 4. Only the end faces of the vaporizer unit 21 are each provided with an electrically conductive coating 4. In this example, the material of the vaporizer unit 21 comprises an electrically conductive additive so that the vaporizer unit 21 is electrically conductive in itself. Thus, the vaporizer unit 21 itself in combination with the two electrically conductive coatings 4 form a heating device 24 which can be connected to a power supply via the electrical contacts 28a, 28b.

The vaporizer units 21 according to FIGS. 13 and 14 are particularly suitable for use in an electronic cigarette 1 according to FIG. 1.

FIG. 15 shows an exemplary embodiment of a vaporizer unit 21 in a longitudinal section and in a side view. The vaporizer unit 21 is formed to be substantially circular cylindrical. In this example, the surface 3 of the vaporizer unit 21 is coated in certain sections with an electrically conductive coating 4. Specifically, the outer surface of the circular cylindrical vaporizer unit 21 is provided with an electrically conductive coating 4. In this example, the electrically conductive coating 4 constitutes a heating device 24 which can be connected to a power supply via electrical contacts 28a, 28b.

FIG. 16 shows another exemplary embodiment of a vaporizer unit 21 in a longitudinal section and in a side view. The vaporizer unit 21 is formed to be substantially circular cylindrical. The material of the vaporizer unit 21 comprises an electrically conductive additive, so that the vaporizer unit 21 is electrically conductive in itself. Two separate, circumferential electrically conductive coatings 4 are applied to the lateral surface of the vaporizer unit 21. In connection with the two electrically conductive coatings 4, the vaporizer unit 21 forms a heating device 24 which can be connected to a power supply via electrical contacts 28a, 28b.

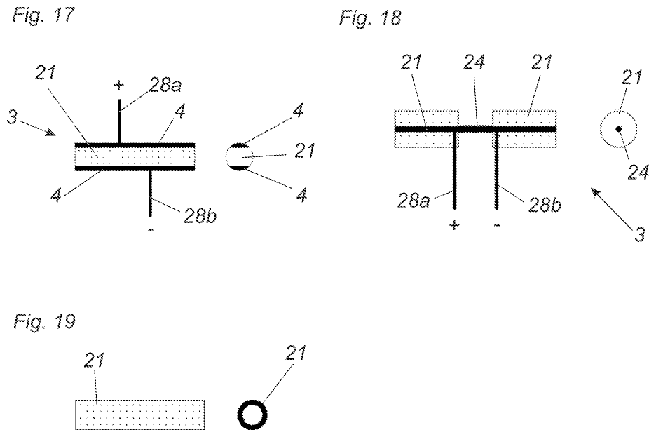

FIG. 17 shows another exemplary embodiment of a vaporizer unit 21 in a longitudinal section and in a side view. The vaporizer unit 21 is formed to be substantially circular cylindrical. The material of the vaporizer unit 21 comprises an electrically conductive additive, so that the vaporizer unit 21 is electrically conductive in itself. Two electrically conductive coatings 4 that are separated from each other and run along a longitudinal extent of the vaporizer unit 21 are applied to the outer surface of the vaporizer unit 21. In connection with the two electrically conductive coatings 4, the vaporizer unit 21 forms a heating device 24 which can be connected to a power supply via electrical contacts 28a, 28b.

FIG. 18 shows an exemplary embodiment of a vaporizer device 2 in a longitudinal section and in a side view. The vaporizer device 2 comprises two circular cylindrical vaporizer units 21 spaced apart from each other in the longitudinal direction and a heating device 24 in the form of an electrically conductive wire which is integrated in both vaporizer units 21 and connects both vaporizer units 21 to each other. The heating device 24 can be connected to a power supply via electrical contacts 28a, 28b.

FIG. 19 shows another exemplary embodiment of a vaporizer unit 21 in a front view and in a side view. In this example, the vaporizer unit 21 is formed as a textile structure in the form of a PEEK fabric. The vaporizer unit 21 has a wick-like structure so that it is able to optimally hold and transport a liquid for an electronic cigarette. In this example, the PEEK fabric is woven from PEEK filament with a diameter of about 38 μm. The round geometry was produced using conventional weaving processes.

The vaporizer units 21 according to FIGS. 15 to 17 and 19 and the vaporizer device 2 according to FIG. 18 are particularly suitable for use in an electronic cigarette 1 according to FIG. 7.

1. A vaporizer unit (21) for a vaporizer device (2) of an inhaler, in particular an electronic cigarette (1), characterized in that the vaporizer unit (21) consists of a material comprising at least one thermoplastic material, preferably a high-performance thermoplastic.

2. The vaporizer unit according to claim 1, characterized in that a melting temperature of the at least one thermoplastic material is greater than about 180° C., preferably greater than about 250° C.

3. The vaporizer unit according to claim 1 or 2, characterized in that a glass transition temperature of the at least one thermoplastic material is greater than about 80° C., preferably greater than about 150° C.

4. The vaporizer unit according to any one of claims 1 to 3, characterized in that the material of the vaporizer unit (21) comprises at least one of the following thermoplastic materials: a polyaryletherketone (PAEK), preferably polyetheretherketone (PEEK); a poly sulfone, preferably polysulfone (PSU) and/or polyethersulfone (PES) and/or polyphenylsulfone (PPSU); a polyimide (PI), preferably polyetherimide (PEI) and/or polyamide-imide (PAI); polyphenylene sulfide (PPS); polyamide (PA); polyphenylene ether (PPE); polyphthalamide (PPA).

5. The vaporizer unit according to any one of claims 1 to 4, characterized in that the vaporizer unit (21) consists substantially entirely of at least one polyaryletherketone, preferably polyetheretherketone.

6. The vaporizer unit according to any one of claims 1 to 5, characterized in that the vaporizer unit (21) has an open porosity of about 30% to 96%, preferably of about 50% to 95%, particularly preferably of about 65% to 94%.

7. The vaporizer unit according to any one of claims 1 to 6, characterized in that the vaporizer unit (21) is self-supporting.

8. The vaporizer unit according to any one of claims 1 to 7, characterized in that the material of the vaporizer unit (21) comprises at least one additive.

9. The vaporizer unit according to any one of claims 1 to 8, characterized in that the material of the vaporizer unit (21) comprises at least one electrically conductive additive, the at least one electrically conductive additive preferably being a metal, graphite, graphene, silicon carbide or activated carbon.

10. The vaporizer unit according to any one of claims 1 to 9, characterized in that the vaporizer unit (21) is formed to be cylindrical, preferably circular cylindrical.

11. The vaporizer unit according to any one of claims 1 to 10, characterized in that a surface (3) of the vaporizer unit (21) is provided at least in certain sections with at least one electrically conductive coating (4).

12. The vaporizer unit according to one of claims 1 to 11, characterized in that the vaporizer unit (21) is formed at least in certain sections, preferably substantially entirely, as a textile structure, preferably as a flat or spatial fabric, the textile structure comprising at least one fiber and/or at least one filament comprising the at least one thermoplastic material.

13. The vaporizer unit according to claim 12, characterized in that the at least one fiber and/or the at least one filament consist or consists substantially entirely of at least one polyaryletherketone, preferably polyetheretherketone.

14. The vaporizer unit according to claim 12 or 13, characterized in that the at least one fiber and/or the at least one filament has or have a diameter of less than 1000 μm, preferably less than 500 μm, particularly preferably less than 300 μm.

15. The vaporizer unit according to any one of claims 12 to 14, characterized in that the textile structure is formed to be wick-like.

16. A vaporizer device (2) for an inhaler, in particular an electronic cigarette (1), having at least one vaporizer unit (21) according to any one of claims 1 to 15.

17. The vaporizer device (2) according to claim 16, characterized in that a heating device (24) is provided, the heating device (24) being arranged in or on the vaporizer unit (21).

18. The vaporizer device according to claim 16 or 17, characterized in that the vaporizer device (2) comprises a receiving chamber (22) for a liquid, the vaporizer unit (21) being in communication with the liquid.

19. An inhaler, in particular electronic cigarette (1), having at least one vaporizer device (2) according to any one of claims 16 to 18.

20. A mouthpiece (30) for an inhaler, in particular an electronic cigarette (1), having at least one vaporizer device (2) according to any one of claims 16 to 18.

21. A method for producing a vaporizer unit (21) according to any one of claims 1 to 11, characterized in that a mixture comprising at least one thermoplastic material, preferably a high-performance thermoplastic, is provided, the mixture being heated until the mixture has melted at least superficially or a melt forms, at least one vaporizer unit (21) being formed from the at least superficially melted mixture or the melt.

22. The method according to claim 21, characterized in that the mixture comprises at least one electrically conductive additive—preferably a metal, graphite, graphene, silicon carbide or activated carbon—which is mixed with the at least one thermoplastic material to provide the mixture.

23. The method according to claim 21 or 22, characterized in that the mixture comprises at least one additive which is mixed with the at least one thermoplastic material to provide the mixture.

24. The method according to claim 23, characterized in that the mixture comprises at least one soluble solid, preferably a salt, the at least one soluble solid being mixed with the at least one thermoplastic material to provide the mixture, wherein after molding the at least one vaporizer unit (21), the at least one soluble solid is at least partially, preferably substantially entirely, dissolved out of the vaporizer unit (21).

25. The method according to any one of claims 21 to 24, characterized in that the at least one thermoplastic material is a powdery or flaky polyaryletherketone, preferably polyetheretherketone.

26. The method according to claim 25, characterized in that the mass fraction of the powdery or flaky polyaryletherketone in the mixture is about 4% to 65%,
preferably about 5% to 55%, particularly preferably about 6% to 45%.

27. The method according to claim 25 or 26, characterized in that powdery polyetheretherketone is mixed with sodium chloride, the mass fraction of the powdery polyetheretherketone in the mixture preferably being about 4% to 65%, particularly preferably about 5% to 55%, more particularly preferably about 6% to 45%, an average particle size of the powdery polyetheretherketone preferably being smaller than about 1000 μm, particularly preferably smaller than about 100 μm.

28. The method according to any one of claims 21 to 27, characterized in that the mixture is heated to a maximum temperature between about 350° C. and about 450° C., preferably to about 370° C., the mixture preferably being exposed to the maximum temperature for a residence time of up to 4 h, particularly preferably about 0.5 h to 1.5 h.

29. The method for producing a vaporizer unit (21) according to any one of claims 12 to 15, characterized in that at least one fiber and/or at least one filament comprising the at least one thermoplastic material are or is processed to form a textile structure, preferably a flat or spatial fabric, the vaporizer unit (21) or a part thereof being molded from the textile structure.

30. The method according to claim 29, characterized in that a thread and/or yarn comprising the at least one fiber and/or the at least one filament are or is processed to form the textile structure.

31. The method according to claim 29 or 30, characterized in that the processing is carried out using at least one of the following methods: spinning, weaving, warp-knitting, knitting, braiding, felting, joining.

32. The method according to any one of claims 29 to 31, characterized in that in the step of molding the vaporizer unit (21), the textile structure is cut to size with a tempered blade.

33. The method according to any one of claims 21 to 32, characterized in that at least one electrically conductive coating (4) is applied at least in certain section onto a surface (3) of the vaporizer unit (21).

34. The method according to claim 33, characterized in that the at least one electrically conductive coating (4) is applied onto the surface (3) of the vaporizer unit (21) by electroplating.

35. The method according to claim 33 or 34, characterized in that the at least one electrically conductive coating (4) is applied onto the surface (3) of the vaporizer unit (21) by vapor deposition.
